(19) United States
(12) Patent Application Publication
(10) Pub. No.: US 2005/0198710 A1
(43) Pub. Date: Sep. 8, 2005

(54) Method of improving soybean protein color by reducing levels of flavonol

(76) Inventors: Brian McGonigle, Wilmington, DE (US); Carl A. Maxwell, Elkton, MD (US); Aideen Oonagh Hession, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/819,773

(22) Filed: Apr. 7, 2004

Related U.S. Application Data
(60) Provisional application No. 60/462,467, filed on Apr. 10, 2003.

(51) Int. Cl. A01H 1/00; C12N 15/82; A01H 5/00; C07H 21/04; C07K 14/415

(52) U.S. Cl. 800/284; 800/312; 435/415; 435/468; 536/23.6; 530/370

(57) ABSTRACT

Recombinant constructs for reducing flavonol levels in a flavonol-producing plants are described. Such constructs comprise a promoter operably linked to a stem-loop structure wherein a nucleic acid sequence of at least 200 nucleotides and having at least 75% sequence identity to SEQ ID NO:4 forms part of loop part of the structure. Methods for reducing flavonol levels are also described.

This application claims the benefit of U.S. Provisional Application No. 60/462,467, filed Apr. 10, 2003. The entire content of this application is herein incorporated by reference.

### FIELD OF THE INVENTION

This invention pertains to methods of reducing flavonol levels in soybean plants and plant parts by transforming plants with a recombinant DNA construct comprising a nucleic acid sequence of at least 200 nucleotides and having at least 75% sequence identity to a polynucleotide encoding a flavonol synthase.

Flavonoids and carotenoids are the two main pigments of plants. Carotenoids are biosynthesized by the terpenoid pathway and are yellow, orange, and red, and include such compounds as alpha-carotene, beta-carotene, lycopene and lutein. Flavonoids are synthesized by the phenylpropanoid pathway and include a wide range of colored substances and include anthocyanins, flavones, chalcones, and aurones. The flavonoids are produced through the phenylpropanoid pathway, a representation of which is shown in FIG. 1. In this pathway phenylalanine ammonia lyase converts phenylalanine to cinnamate; cinnamic acid hydroxylase converts cinnamate to p-coumarate; and coumarate:CoA ligase converts p-coumarate to p-coumaroyl-CoA. Lignins may be produced from p-coumaroyl-CoA or from p-coumarate.

The first committed step in the formation of flavonoids is catalyzed by chalcone synthase (CHS). This step consists of the condensation of one molecule of p-coumaroyl-CoA and three molecules of malonyl-CoA to form 4,2′,4′,6′-tetrahydroxychalcone. In certain species, a chalcone synthase together with a chalcone reductase and NADPH as a cofactor generate isoliquiritigenin, a 6-deoxychalcone. In a reaction catalyzed by chalcone isomerase, isoliquiritigenin is isomerized to form liquiritigenin that is the precursor to daidzein, glycitein, and the pterocarpan phytoalexins. Also catalyzed by chalcone isomerase is the isomerization of 4,2′,4′,6′-tetrahydroxychalcone (naringenin chalcone) to naringenin (5,7,4′-trihydroxyflavanone), the precursor to genistein, anthocyanins, flavones, flavonols, and others.

Isoflavone synthase catalyzes the transformation of naringenin to the unstable intermediate 2-hydroxyisoflavanone which is converted to genistein. Naringenin may also be dehydrated by the action of flavone synthase to produce flavones such as apigenin. Naringenin may be hydroxylated by flavanone 3-hydroxylase to the dihydroflavonol dihydrokaempferol. Dihydroflavonols are the precursors of anthocyanins, flavonols, and condensed tannins. Further hydroxylation of dihydrokaempferol by flavanone 3′ hydroxylase results in the formation of dihydroquercetin. Additionally, dihydrokaempferol or dihydroquercetin can be further hydroxylated by flavanone 3′, 5′ hydroxylase to form dihydromyricetin. Flavonol synthase (FLS) catalyses the conversion of the dihydroflavonols dihydrokaempferol, dihydroquercetin, dihydromyricetin to the flavonols kaempferol, quercetin, and myricetin respectively. In most plants anthocyanins and flavonols are synthesized within the same cell and usually accumulate in the same subcellular location (Holton, T. A. et al. (1995) Plant Cell 7:1071-1083). FLS is a soluble dioxygenase that requires 2-oxoglutarate, ferrous ion, and ascorbate for full activity (Britsch, L (1990) Arch. Biochem. Biophys. 282:152-160).

Flavonol synthase cDNAs have been identified from petunia (Holton, T. A. et al. (1993) Plant J. 4:1003-1010), Arabidopsis thaliana (Wisman, E. et al. (1998) Proc. Natl. Acad. Sci. U.S.A. 95:12432-12437), Eustoma grandiflorum and Solanum tuberosum (van Eldik, G. J. et al. (1997) Plant J. 11:105-113), Malus×domestica (NCBI General identifier No. 4588916), and Citrus unshiu (Wellmann, F. et al. (2002) Eur. J. Biochem. 269:4134-4142), among others. Soybean sequences encoding an entire FLS have been identified, as have sequences encoding portions of Zea mays, Momordica charantia, Limnanthes douglasii, and Triticum aestivum FLS (U.S. Pat. No. 6,380,464).

The use of fragments encoding flavonol synthases for altering the levels of flavonols has been suggested. It is mentioned in U.S. Pat. No. 6,380,464 that the polynucleotides of the invention may be used to alter the levels of flavonol synthase in a cell. Manipulation of the levels of flavonol synthase has been used in an effort to manipulate the petal color of petunia and tobacco flowers (U.S. Pat. No. 5,859,329). It is described in this patent that transgenic petunia plants were prepared with constructs containing a portion of the petunia FLS coding region in sense or anti sense orientation with respect to the promoter. Some of these plants produced redder flowers and were found to produce markedly less flavonols than non-transgenic plants. It was concluded that this was an indication of the reduction in flavonol production due to the suppression of FLS activity. Transgenic tobacco plants prepared using the antisense construct also produced redder flowers. The red flower color In this case was attributed to a three-fold increase in anthocyanin production in the corolla limb. The use of hairpin-mediated suppression is not mentioned in either of these two U.S. patents.

Because flavonols are colored compounds that may accumulate in the seed, manipulation of the flavonol synthase levels may affect the color of the seed produced. In turn foods produced from seeds with altered levels of flavonols may be of different colors than foods made from seeds of the wild type counterparts. The color of food is an important determinant in how people perceive that food. People expect certain colors for certain foods. By altering the levels of flavonols in soybean we can produce milk from said soybean that will be whiter and hence more appealing to consumers.

### SUMMARY OF THE INVENTION

In a first embodiment the invention concerns a recombinant construct for reducing the level of flavonol in a flavonol-producing plant which comprises a promoter operably linked to a stem-loop structure wherein a nucleic acid sequence of at least 200 nucleotides and having at least 75% identity to SEQ ID NO:4 forms the loop part of the structure. Preferably, the promoter is a seed specific promoter. More preferably, the promoter is selected from the group consisting of a β-conglycinin promoter, a napin promoter, and a phaseolin promoter. Most preferably, the stem of the stem-loop structure consists essentially of SEQ ID NO:6.

In a second embodiment, the invention concerns a flavonol-producing plant comprising in its genome a recombinant construct comprising a promoter operably linked to a stem-loop structure wherein a nucleic acid sequence of at least 200 nucleotides and having at least 75% identity to SEQ ID NO:4 forms the loop part of the structure and wherein said plant has a reduced level of flavonol as compared to a plant which does not comprise the recombinant DNA construct. It is preferred that the flavonol-producing plant is soybean. Seeds and plant parts having a reduced flavonol level due to the presence of the recombinant construct of the invention are included.

In a third embodiment, the invention concerns a protein product having improved color wherein the product is obtained from the seeds or plant parts of the invention. In a preferred embodiment the product is selected from the group consisting of protein isolate, protein concentrate, meal, grits, full fat and defatted flours, textured proteins, textured flours, textured concentrates, and textured isolates.

In another embodiment the invention includes a food product having incorporated the protein product having improved color of the invention. The food product may be selected from a dairy product, a food bar, a nutritional supplement, or a beverage.

Also included within the invention is a method of reducing flavonol levels in soybean plants and plant parts by transforming such plants or plant parts with a recombinant DNA construct comprising a nucleic acid sequence of at least 200 nucleotides and having at least 75% sequence identity with a polynucleotide encoding a flavonol synthase.

### BRIEF DESCRIPTION OF THE DRAWINGS AND SEQUENCE LISTINGS

The invention can be more fully understood from the following detailed description and the accompanying drawings and Sequence Listing which form a part of this application.

FIG. 1 depicts the phenylpropanoid pathway for the production of flavonols. FIG. 1A shows the pathway from phenylalanine to glycitein, daidzein, phytoalexins, and naringenin. FIG. 1B shows the further metabolism of naringenin to flavonols, flavanones, and condensed tannins.

FIG. 2 depicts a representation of the seed-specific expression vector pKS151. The two copies of the 36-nucleotide sequence are indicated by “EL”, and the inverted-repeat of the 36-nucleotide sequences is indicated by “(EL)”.

FIG. 3 depicts the μg kaempferol per gram soybean observed for bulk R1 seeds from transformed plants containing plasmid AC24 and from control plants. For ease of understanding, the plants from which the seeds are derived are numbered 1 through 88 in the figure and the plant number indicated in Table 2. FIG. 3A presents the results obtained for plants 144. FIG. 3B presents the results obtained for plants 45-88. Plants 77-88 are non-transgenic, with plants 77-82 being from 93B41 and plants 83-88 being from Jack.

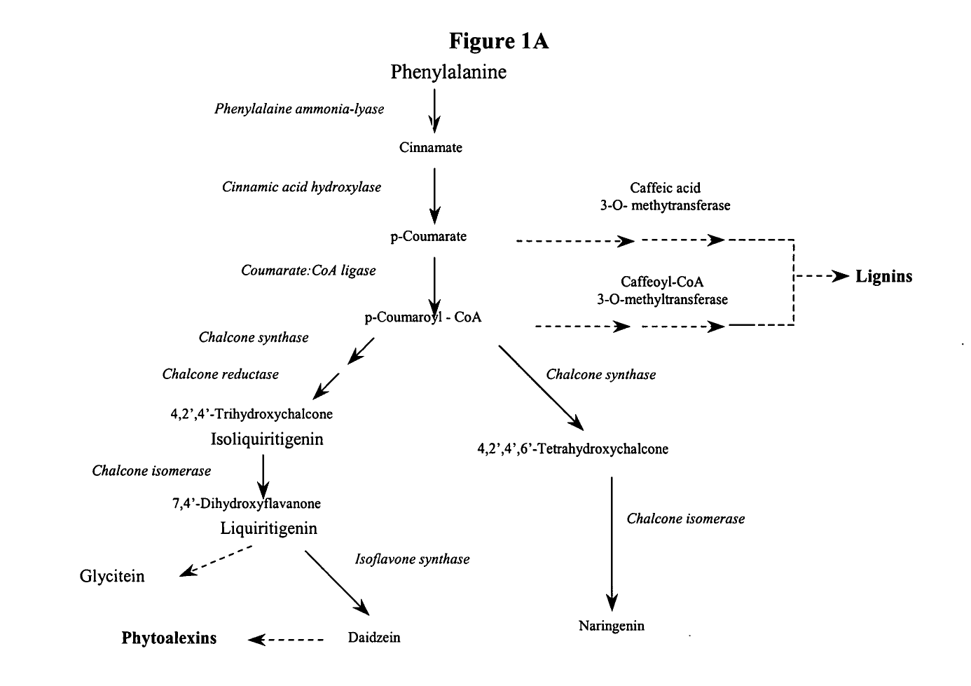

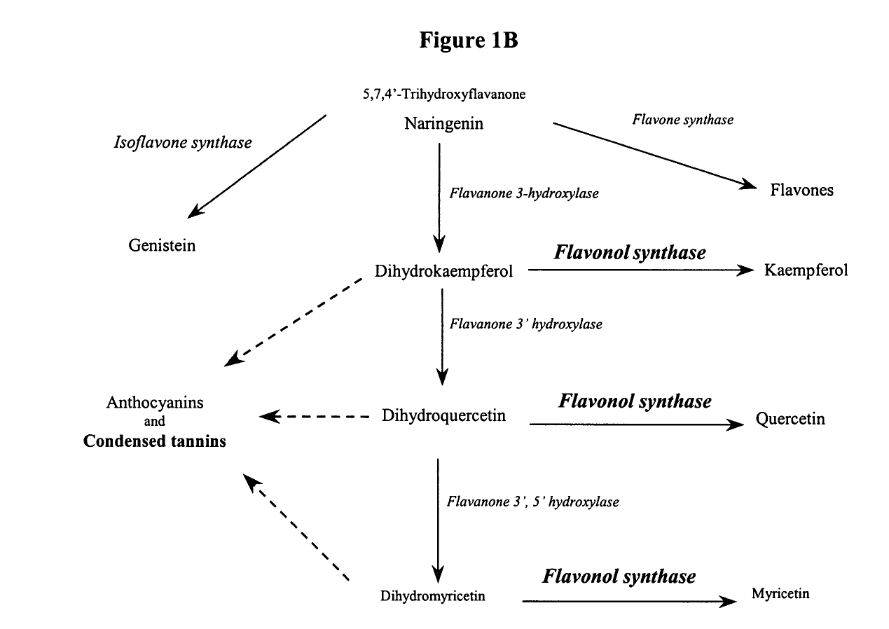

The following sequence descriptions and Sequences Listing attached hereto comply with the rules governing nucleotide and/or amino acid sequence disclosures in patent applications as set forth in 37 C.F.R. §1.821-1.825. The Sequence Listing contains the one letter code for nucleotide sequence characters and the three letter codes for amino acids as defined in conformity with the IUPAC-IUB standards described in Nucleic Acids Research 13:3021-3030 (1985) and in the Biochemical Journal 219 (No. 2):345-373 (1984) which are herein incorporated by reference. The symbols and format used for nucleotide and amino acid sequence data comply with the rules set forth in 37 C.F.R. §1.822.

SEQ ID NO:1 is the nucleotide sequence of plasmid pKS151 used for the expression of a fragment of flavonol synthase.

SEQ ID NO:2 is the nucleotide sequence of primer flavonol synthase-Not1-sense used to amplify a portion of a flavonol synthase from clone ssl.pk0057.d12.

SEQ ID NO:3 is the nucleotide sequence of primer flavonol-Not1-antisense used to amplify a portion of a flavonol synthase from clone ssl.pk0057.d12.

SEQ ID NO:4 is the nucleotide sequence of the entire cDNA insert in clone ssl.pk0057.d12 encoding a flavonol synthase.

SEQ ID NO:5 is the nucleotide sequence of primer Fsh-A used to detect the presence of the flavonol synthase construct in transgenic plants.

SEQ ID NO:6 is the nucleotide sequence of primer Ps17 used to detect the presence of the flavonol synthase construct in transgenic plants.

SEQ ID NO:7 is the nucleotide sequence of nucleotides 5451-5567 from SEQ ID NO:1. This nucleotide sequence corresponds to the polynucleotide fragment consisting of a unique Not 1 restriction endonuclease site surrounded by nucleotides that promote formation of a stem structure which are flanked by Eag I restriction endonuclease sites.

### DETAILED DESCRIPTION OF THE INVENTION

All patents, patent applications, and publications cited are incorporated herein by reference in their entirety.

In the context of this disclosure, a number of terms shall be utilized.

The term “kaempferol” refers to the flavonol that results from the reaction of flavonol synthase with dihydrokaempferol.

The term “quercetin” refers to the flavonol that results from the reaction of flavonol synthase with dihydroquercetin.

The term “flavonol synthase” refers an enzyme that catalyzes the formation of a flavonol from a dihydroflavonol. The flavonol formed includes, but is not limited to, kaempferol or quercetin. Flavonols may exist in a free form or in a bound form conjugated to sugars or organic acids.

The term “flavonol-producing plant” refers to a plant that is normally capable of producing flavonols. It is preferred that the flavonol-producing plant of the invention be a soybean plant.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The terms “subfragment that is functionally equivalent” and “functionally equivalent subfragment” are used interchangeably herein. These terms refer to a portion or subsequence of an isolated nucleic acid fragment in which the ability to alter gene expression or produce a certain phenotype is retained whether or not the fragment or subfragment encodes an active enzyme. For example, the fragment or subfragment can be used in the design of chimeric genes to produce the desired phenotype in a transformed plant. Chimeric genes can be designed for use in suppression by linking a nucleic acid fragment or subfragment thereof, whether or not it encodes an active enzyme, in the sense or antisense orientation relative to a plant promoter sequence.

The terms “homology”, “homologous”, “substantially similar” and “corresponding substantially” are used interchangeably herein. They refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

Moreover, the skilled artisan recognizes that substantially similar nucleic acid sequences encompassed by this invention are also defined by their ability to hybridize, under moderately stringent conditions (for example, 0.5×SSC, 0.1% SDS, 60° C.) with the sequences exemplified herein, or to any portion of the nucleotide sequences disclosed herein and which are functionally equivalent to any of the nucleic acid sequences disclosed herein. Stringency conditions can be adjusted to screen for moderately similar fragments, such as homologous sequences from distantly related organisms, to highly similar fragments, such as genes that duplicate functional enzymes from closely related organisms. Post-hybridization washes determine stringency conditions. One set of preferred conditions involves a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 minutes. A more preferred set of stringent conditions involves the use of higher temperatures in which the washes are identical to those above except for the temperature of the final two 30 min washes in 0.2×SSC, 0.5% SDS was increased to 60° C. Another preferred set of highly stringent conditions involves the use of two final washes in 0.1×SSC, 0.1% SDS at 65° C.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” refers any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure. An “allele” is one of several alternative forms of a gene occupying a given locus on a chromosome. When all the alleles present at a given locus on a chromosome are the same that plant is homozygous at that locus. If the alleles present at a given locus on a chromosome differ that plant is heterozygous at that locus.

“Coding sequence” refers to a DNA sequence that codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

“Promoter” refers to a DNA sequence capable of controlling the expression of a coding sequence or functional RNA. The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. It is further recognized that since in most cases the exact boundaries of regulatory sequences have not been completely defined, DNA fragments of some variation may have identical promoter activity. Promoters that cause a gene to be expressed in most cell types at most times are commonly referred to as “constitutive promoters”. New promoters of various types useful in plant cells are constantly being discovered; numerous examples may be found in the compilation by Okamuro, J. K., and Goldberg, R. B. (1989) Biochemistry of Plants 15:1-82.

The “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D. (1995) Mol. Biotechnol. 3:225-236).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al. (1989) Plant Cell 1:671-680.

“RNA transcript” refers to the product resulting from RNA polymerase-catalyzed transcription of a DNA sequence. When the RNA transcript is a perfect complementary copy of the DNA sequence, it is referred to as the primary transcript. An RNA transcript is referred to as the mature RNA when it is an RNA sequence derived from post-transcriptional processing of the primary transcript. “Messenger RNA (mRNA)” refers to the RNA that is without introns and that can be translated into protein by the cell. “cDNA” refers to a DNA that is complementary to and synthesized from a mRNA template using the enzyme reverse transcriptase. The cDNA can be single-stranded or converted into the double-stranded form using the Klenow fragment of DNA polymerase I. “Sense” RNA refers to RNA transcript that includes the mRNA and can be translated into protein within a cell or in vitro. “Antisense RNA” refers to an RNA transcript that is complementary to all or part of a target primary transcript or mRNA, and that blocks the expression of a target gene (U.S. Pat. No. 5,107,065). The complementarity of an antisense RNA may be with any part of the specific gene transcript, i.e., at the 5′ non-coding sequence, 3′ non-coding sequence, introns, or the coding sequence. “Functional RNA” refers to antisense RNA, ribozyme RNA, or other RNA that may not be translated but yet has an effect on cellular processes. The terms “complement” and “reverse complement” are used interchangeably herein with respect to mRNA transcripts, and are meant to define the antisense RNA of the message.

The term “operably linked” refers to the association of nucleic acid sequences on a single nucleic acid fragment so that the function of one is regulated by the other. For example, a promoter is operably linked with a coding sequence when it is capable of regulating the expression of that coding sequence (i.e., that the coding sequence is under the transcriptional control of the promoter). Coding sequences can be operably linked to regulatory sequences in a sense or antisense orientation. In another example, the complementary RNA regions of the invention can be operably linked, either directly or indirectly, 5′ to the target mRNA, or 3′ to the target mRNA, or within the target mRNA, or a first complementary region is 5′ and its complement is 3′ to the target mRNA.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989. Transformation methods are well known to those skilled in the art and are described below.

“PCR” or “Polymerase Chain Reaction” is a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

The term “recombinant” refers to an artificial combination of two otherwise separated segments of sequence, e.g., by chemical synthesis or by the manipulation of isolated segments of nucleic acids by genetic engineering techniques.

The terms “recombinant construct”, “expression construct”, “chimeric construct”, “construct”, and “recombinant DNA construct” are used interchangeably herein. A recombinant construct comprises an artificial combination of nucleic acid fragments, e.g., regulatory and coding sequences that are not found together in nature. For example, a chimeric construct may comprise regulatory sequences and coding sequences that are derived from different sources, or regulatory sequences and coding sequences derived from the same source, but arranged in a manner different than that found in nature. Such construct may be used by itself or may be used in conjunction with a vector. If a vector is used then the choice of vector is dependent upon the method that will be used to transform host cells as is well known to those skilled in the art. For example, a plasmid vector can be used. The skilled artisan is well aware of the genetic elements that must be present on the vector in order to successfully transform, select and propagate host cells comprising any of the isolated nucleic acid fragments of the invention. The skilled artisan will also recognize that different independent transformation events will result in different levels and patterns of expression (Jones et al., (1985) EMBO J. 4:2411-2418; De Almeida et al., (1989) Mol. Gen. Genetics 218:78-86), and thus that multiple events must be screened in order to obtain lines displaying the desired expression level and pattern. Such screening may be accomplished by Southern analysis of DNA, Northern analysis of mRNA expression, immunoblotting analysis of protein expression, or phenotypic analysis, among others.

The term “expression”, as used herein, refers to the production of a functional end-product e.g., a mRNA or a protein (precursor or mature).

“Mature” protein refers to a post-translationally processed polypeptide; i.e., one from which any pre- or propeptides present in the primary translation product have been removed. “Precursor” protein refers to the primary product of translation of mRNA; i.e., with pre- and propeptides still present. Pre- and propeptides may be but are not limited to intracellular localization signals.

“Stable transformation” refers to the transfer of a nucleic acid fragment into a genome of a host organism, including both nuclear and organellar genomes, resulting in genetically stable inheritance. In contrast, “transient transformation” refers to the transfer of a nucleic acid fragment into the nucleus, or DNA-containing organelle, of a host organism resulting in gene expression without integration or stable inheritance. Host organisms containing the transformed nucleic acid fragments are referred to as “transgenic” organisms.

“Antisense inhibition” refers to the production of antisense RNA transcripts capable of suppressing the expression of the target protein. “Co-suppression” refers to the production of sense RNA transcripts capable of suppressing the expression of identical or substantially similar foreign or endogenous genes (U.S. Pat. No. 5,231,020). Co-suppression constructs in plants previously have been designed by focusing on overexpression of a nucleic acid sequence having homology to an endogenous mRNA, in the sense orientation, which results in the reduction of all RNA having homology to the overexpressed sequence (see Vaucheret et al. (1998) Plant J. 16:651-659; and Gura (2000) Nature 404:804-808). The overall efficiency of this phenomenon is low, and the extent of the RNA reduction is widely variable.

Recent work has described the use of “hairpin” structures that incorporate all, or part, of an mRNA encoding sequence in a complementary orientation that results in a potential “stem-loop” structure for the expressed RNA (PCT Publication WO 99/53050 published on Oct. 21, 1999 and more recently, Applicants' assignee's own WO 02/00904 published on Jan. 3, 2002). Hairpin structures may contain the target RNA forming either the stem or the loop of the hairpin. Another variation describes the use of plant viral sequences to direct the suppression, or “silencing”, of proximal mRNA encoding sequences (PCT Publication WO 98/36083 published on Aug. 20, 1998). Both of these co-suppressing phenomena have not been elucidated mechanistically, although genetic evidence has begun to unravel this complex situation (Elmayan et al. (1998) Plant Cell 10:1747-1757).

The polynucleotide sequences used for suppression do not necessarily have to be 100% complementary to the polynucleotide sequences found in the gene to be suppressed. For example, suppression of all the subunits of the soybean seed storage protein β-conglycinin has been accomplished using a polynucleotide derived from a portion of the gene encoding the α subunit (U.S. Pat. No. 6,362,399). β-conglycinin is a heterogeneous glycoprotein composed of varying combinations of three highly negatively charged subunits identified as α, α′ and β. The polynucleotide sequences encoding the α and α′ subunits are 85% identical to each other while the polynucleotide sequences encoding the β subunit are 75 to 80% identical to the α and α′ subunits. Thus, polynucleotides that are at least 75% identical to a region of the polynucleotide that is target for suppression have been shown to be effective in suppressing the desired target. The polynucleotide should be at least 80% identical, preferably at least 90% identical, most preferably at least 95% identical, or the polynucleotide may be 100% identical to the desired target.

Any promoter can be used in accordance with the method of the invention. Thus, the origin of the promoter chosen to drive expression of the coding sequence is not important as long as it has sufficient transcriptional activity to accomplish the invention by expressing translatable mRNA for the desired nucleic acid fragments in the desired host tissue. Either heterologous or non-heterologous (i.e., endogenous) promoters can be used to practice the invention. The promoter for use in the present invention may be selected from the group consisting of a seed-specific promoter, root-specific promoter, vacuole-specific promoter, and an embryo-specific promoter.

Among the most commonly used promoters are the nopaline synthase (NOS) promoter (Ebert et al. (1987) Proc. Natl. Acad. Sci. U.S.A. 84:5745-5749), the octapine synthase (OCS) promoter, caulimovirus promoters such as the cauliflower mosaic virus (CaMV) 19S promoter (Lawton et al. (1987) Plant Mol. Biol. 9:315-324), the CaMV 35S promoter (Odell et al. (1985) Nature 313:810-812), and the figwort mosaic virus 35S promoter, the light inducible promoter from the small subunit of rubisco, the Adh promoter (Walker et al. (1987) Proc. Natl. Acad. Sci. U.S.A. 84:6624-66280, the sucrose synthase promoter (Yang et al. (1990) Proc. Natl. Acad. Sci. U.S.A. 87:4144-4148), the R gene complex promoter (Chandler et al. (1989) Plant Cell 1:1175-1183), the chlorophyll a/b binding protein gene promoter, etc. Other commonly used promoters are, the promoters for the potato tuber ADPGPP genes, the sucrose synthase promoter, the granule bound starch synthase promoter, the glutelin gene promoter, the maize waxy promoter, Brittle gene promoter, and Shrunken 2 promoter, the acid chitinase gene promoter, and the zein gene promoters (15 kD, 16 kD, 19 kD, 22 kD, and 27 kD; Perdersen et al. (1982) Cell 29:1015-1026). A plethora of promoters is described in WO 00/18963, published on Apr. 6, 2000, the disclosure of which is hereby incorporated by reference.

Examples of a seed-specific promoter include, but are not limited to, the promoter for β-conglycinin (Chen et al. (1989) Dev. Genet. 10: 112-122), the napin promoter, and the phaseolin promoter. Other tissue-specific promoters that may be used to accomplish the invention include, but are not limited to, the chloroplast glutamine synthase (GS2) promoter (Edwards et al. (1990) Proc. Natl. Acad. Sci. U.S.A. 87:3459-3463), the chloroplast fructose-1,6-biophosphatase promoter (Lloyd et al. (1991) Mol. Gen. Genet. 225:209-2216), the nuclear photosynthetic (ST-LS1) promoter (Stockhaus et al. (1989) EMBO J. 8:2445-2451), the serine/threonine kinase (PAL) promoter, the glucoamylase promoter, the promoters for the Cab genes (cab6, cab-1, and cab-1R, Yamamoto et al. (1994) Plant Cell Physiol. 35:773-778; Fejes et al. (1990) Plant Mol. Biol. 15:921-932; Lubberstedt et al. (1994) Plant Physiol. 104:997-1006; Luan et al. (1992) Plant Cell 4:971-981), the pyruvate orthophosphate dikanase promoter (Matsuoka et al. (1993) Proc. Natl. Acad. Sci. U.S.A. 90:9586-9590), the LhcB promoter (Cerdan et al. (1997) Plant Mol. Biol. 33:245-255), the PsbP promoter (Kretsch et al. (1995) Plant Mol. Biol. 28:219-229), the SUC2 sucrose H+ symporter promoter (Truernit et al. (1995) Planta 196:564-570), and the promoters for the thylakoid membrane genes (psaD, psaF, psaE, PC, FNR, atpC, atpD), etc.

Suppression of the expression and/or function of a gene in general is described above. However, in a preferred embodiment, the recombinant construct comprises a stem-loop structure as described in PCT publication WO 02/00904, published Jan. 3, 2002. WO 02/00904 discloses that suitable nucleic acid sequences and their reverse complement can be used to alter the expression of any homologous, endogenous RNA (i.e., the target RNA) which is flanked by the suitable nucleic acid sequence and its reverse complement. The suitable nucleic acid sequence and its reverse complement can be unrelated to any endogenous RNA in the host, can be transcribed for by any nucleic acid sequence in the genome of the host provided that nucleic acid sequence is not transcribed to any target mRNA or any sequence that is substantially similar to the target mRNA, or can be translated into a synthetic or non-naturally occurring polypeptide. What is presented in WO 02/00904 is a very efficient and robust approach to achieving single, or multiple, gene co-suppression using single plasmid transformation. Such constructs are composed of promoters linked to mRNA(s) coding regions, or fragments thereof, that are targeted for suppression, and short complementary sequences that are unrelated to the targets. The complementary sequences can be oriented both 5′, both 3′, or on either side of the target sequence. The complementary sequences are preferred to be about 40-50 nucleotides in length, or more preferably 50-100 nucleotides in length, or most preferably at least or greater than 100-300 nucleotides.

The complementary sequences are unrelated to the target, but can come from any other source. Preferred embodiments of these sequences include, but are not limited to, plant sequences, bacterial sequences, animal sequences, viral or phage sequences, or completely artificial, i.e. non-naturally occurring, sequences not known to occur in any organism. These complementary sequences can be synthesized using conventional means well known to those skilled in the art. Non-naturally complementary regions which can be used to practice the invention include, but are not limited to, a polynucleotide encoding the polypeptide Glu Leu Val Ile Ser Leu Ile Val Glu Ser (“ELVISLIVES”).

Methods for transforming dicots, primarily by use of Agrobacterium tumefaciens, and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brassica (U.S. Pat. No. 5,463,174); peanut (Cheng et al. (1996) Plant Cell Rep. 15:653-657, McKently et al. (1995) Plant Cell Rep. 14:699-703); papaya (Ling, K. et al. (1991) Bio/technology 9:752-758); and pea (Grant et al. (1995) Plant Cell Rep. 15:254-258). For a review of other commonly used methods of plant transformation see Newell, C. A. (2000) Mol. Biotechnol. 16:53-65. One of these methods of transformation uses Agrobacterium rhizogenes (Tepfler, M. and Casse-Delbart, F. (1987) Microbiol. Sci. 4:24-28). Transformation of soybeans using direct delivery of DNA has been published using PEG fusion (PCT publication WO 92/17598), electroporation (Chowrira, G. M. et al. (1995) Mol. Biotechnol. 3:17-23; Christou, P. et al. (1987) Proc. Natl. Acad. Sci. U.S.A. 84:3962-3966), microinjection, or particle bombardment (McCabe, D. E. et. al. (1988) Bio/Technology 6:923; Christou et al. (1988) Plant Physiol. 87:671-674).

There are a variety of methods for the regeneration of plants from plant tissue. The particular method of regeneration will depend on the starting plant tissue and the particular plant species to be regenerated. The regeneration, development and cultivation of plants from single plant protoplast transformants or from various transformed explants is well known in the art (Weissbach and Weissbach, (1988) In: Methods for Plant Molecular Biology, (Eds.), Academic Press, Inc., San Diego, Calif.). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook et al. (1989) Molecular Cloning: A Laboratory Manual, Cold Spring Harbor Press; Maliga et al. (1995) Methods in Plant Molecular Biology, Cold Spring Harbor Press; Birren et al. (1998) Genome Analysis: Detecting Genes, 1, Cold Spring Harbor, N.Y.; Birren et al. (1998) Genome Analysis: Analyzing DNA, 2, Cold Spring Harbor, N.Y.; Plant Molecular Biology: A Laboratory Manual, eds. Clark, Springer, N.Y. (1997)).

Also within the scope of this invention are seeds or plant parts obtained from such transformed plants. Plant parts include differentiated and undifferentiated tissues, including, but not limited to, roots, stems, shoots, leaves, pollen, seeds, tumor tissue, and various forms of cells and culture such as single cells, protoplasts, embryos, and callus tissue. The plant tissue may be in plant or in organ, tissue or cell culture.

In another aspect, this invention concerns a soybean protein product low in flavonol obtained from the seeds or plant parts obtained from the transformed plants described herein. Besides full-fat flour and partially deflated extracted-expelled flour soybean protein products are derived from defatted flakes. Examples of such a soybean protein product include, but are not limited to, protein isolate, protein concentrate, meal, grits, full fat and defatted flours, textured proteins, textured flours, textured concentrates and textured isolates. In still another aspect, this invention concerns a soybean protein product extracted from the seeds or plant parts obtained from the transformed plants described herein.

“Soy protein products” can be defined as those items produced from seed or other plant part of a suitable soybean which are used in feeds, foods and/or beverages. For example, “soy protein products” can include, but are not limited to, those items listed in Table 1. “Soy protein products”.

TABLE 1
Soy Protein Products Derived from Soybean Seeds^{a}

Whole Soybean Products
Roasted Soybeans
Baked Soybeans
Soy Sprouts
Soy Milk
Specialty Soy Foods/Ingredients
Soy Milk
Tofu
Tempeh
Miso
Soy Sauce
Hydrolyzed Vegetable Protein
Whipping Protein
Processed Soy Protein Products
Full Fat and Defatted Flours
Soy Grits
Soy Hypocotyls
Soybean Meal
Soy Milk
Soy Protein Isolates
Soy Protein Concentrates
Textured Soy Proteins
Textured Flours and Concentrates
Textured Concentrates
Textured Isolates
^{a}See Soy Protein Products: Characteristics, Nutritional Aspects and Utilization (1987). Soy Protein Council.

Methods for obtaining such products are well known to those skilled in the art. For example, in the case of soybean, such products can be obtained in a variety of ways. Conditions typically used to prepare soy protein isolates have been described by [Cho, et al, (1981) U.S. Pat. No. 4,278,597; Goodnight, et al. (1978) U.S. Pat. No. 4,072,670]. Soy protein concentrates are produced by three basic processes: acid leaching (at about pH 4.5), extraction with alcohol (about 55-80%), and denaturing the protein with moist heat prior to extraction with water. Conditions typically used to prepare soy protein concentrates have been described by Pass [(1975) U.S. Pat. No. 3,897,574] and Campbell et al. [(1985) in New Protein Foods, ed. by Altschul and Wilcke, Academic Press, Vol. 5, Chapter 10, Seed Storage Proteins, pp 302-338].

It is common to refer to “food” as that matter suitable for human consumption and to “feed” as that matter suitable for consumption by livestock. A food product, then, is a product consumed by humans.

A “dairy product” includes, but is not limited to, a milk, a cream, a cheese, a butter, or any product derived from the milk of an animal or derived an alternative source in order to mimic those products derived from the milk of an animal. For example a dairy product such as margarine spreads or cheeses may also be prepared from vegetable or other means to mimic the flavor and consistency of its animal-milk-derived counterpart.

A “food bar” is a type of food made in a bar shape or any other shape.

A “nutritional supplement” is a product that is intended to supplement the diet.

A “beverage” is a liquid, different than water, which is used for drinking. A beverage of the invention may be available as a liquid or as a powder that may be dissolved.

“Processing” refers to any physical and chemical methods used to obtain the products listed in Table 1 and includes, but is not limited to, heat conditioning, flaking and grinding, extrusion, solvent extraction, or aqueous soaking and extraction of whole or partial seeds. Furthermore, “processing” includes the methods used to concentrate and isolate soy protein from whole or partial seeds, as well as the various traditional Oriental methods in preparing fermented soy food products. Trading Standards and Specifications have been established for many of these products (see National Oilseed Processors Association Yearbook and Trading Rules 1991-1992). Products referred to as being “high protein” or “low protein” are those as described by these Standard Specifications. “NSI” refers to the Nitrogen Solubility Index as defined by the American Oil Chemists' Society Method Ac4 41. “KOH Nitrogen Solubility” is an indicator of soybean meal quality and refers to the amount of nitrogen soluble in 0.036 M KOH under the conditions as described by Araba and Dale [(1990) Poult. Sci. 69:76-83]. “White” flakes refer to flaked, dehulled cotyledons that have been defatted and treated with controlled moist heat to have an NSI of about 85 to 90. This term can also refer to a flour with a similar NSI that has been ground to pass through a No. 100 U.S. Standard Screen size. “Cooked” refers to a soy protein product, typically a flour, with an NSI of about 20 to 60. “Toasted” refers to a soy protein product, typically a flour, with an NSI below 20. “Grits” refer to defatted, dehulled cotyledons having a U.S. Standard screen size of between No. 10 and 80. “Soy Protein Concentrates” refer to those products produced from dehulled, defatted soybeans by three basic processes: acid leaching (at about pH 4.5), extraction with alcohol (about 55-80%), and denaturing the protein with moist heat prior to extraction with water. Conditions typically used to prepare soy protein concentrates have been described by Pass [(1975) U.S. Pat. No. 3,897,574; Campbell et al., (1985) in New Protein Foods, ed. by Altschul and Wilcke, Academic Press, Vol. 5, Chapter 10, Seed Storage Proteins, pp 302-338]. “Extrusion” refers to processes whereby material (grits, flour or concentrate) is passed through a jacketed auger using high pressures and temperatures as a means of altering the texture of the material. “Texturing” and “structuring” refer to extrusion processes used to modify the physical characteristics of the material. The characteristics of these processes, including thermoplastic extrusion, have been described previously [Atkinson (1970) U.S. Pat. No. 3,488,770, Horan (1985) In New Protein Foods, ed. by Altschul and Wilcke, Academic Press, Vol. 1A, Chapter 8, pp 367-414]. Moreover, conditions used during extrusion processing of complex foodstuff mixtures that include soy protein products have been described previously [Rokey (1983) Feed Manufacturing Technology III, 222-237; McCulloch, U.S. Pat. No. 4,454,804].

Also, within the scope of this invention are food, food supplements, food bars, and beverages that have incorporated therein a flavonol-containing product of the invention. The beverage can be in a liquid or in a dry powdered form.

The flavonol-containing product made according to the process of the present invention can be incorporated into a wide variety of food and beverage applications. For example, it can be integrated into meats such as ground meats, emulsified meats, marinated meats, and meats injected with the soy product of the invention; it can also be incorporated into nutritional supplements; beverages such as nutritional beverages, sports beverages, protein fortified beverages, juices, milk, milk alternatives, and weight loss beverages; cheeses such as hard and soft cheeses, cream cheese, and cottage cheese; frozen desserts such as ice cream, ice milk, low fat frozen desserts, and non-dairy frozen desserts; yogurts; soups; puddings; bakery products; and salad dressings; and dips and spreads such as mayonnaise; and chip dips; and food bars.

The flavonol-containing product of the invention may also be incorporated into a cereal food product, a snack food product, a baked good product, a fried food product, a health food product, an infant formula, a beverage, a nutritional supplement, a dairy product, a pet food product, or animal feed.

A cereal food product is a food product derived from the processing of a cereal grain. A cereal grain includes any plant from the grass family that yields an edible grain (seed). The most popular grains are barley, corn, millet, oats, quinoa, rice, rye, sorghum, triticale, wheat and wild rice. Examples of a cereal food product include, but are not limited to, whole grain, crushed grain, grits, flour, bran, germ, breakfast cereals, extruded foods, pastas, and the like.

A baked good product comprises any of the cereal food products mentioned above and has been baked or processed in a manner comparable to baking, i.e., to dry or harden by subjecting to heat. Examples of a baked good product include, but are not limited to bread crumbs, baked snacks, mini-biscuits, mini-crackers, mini-cookies, and mini-pretzels.

A snack food product comprises any of the above or below described food products.

A fried food product comprises any of the above or below described food products that has been fried.

A health food product is any food product that imparts a health benefit. Many oilseed-derived food products may be considered as health foods.

The beverage can be in a liquid or in a dry powdered form.

For example, there can be mentioned non-carbonated drinks; carbonated drinks; fruit juices, fresh, frozen, canned or concentrate; still or sparkling water; flavored or plain milk drinks, etc. Adult and infant nutritional formulas are well known in the art and commercially available (e.g., Similac®, Ensure®, Jevity®, and Alimentum® from Ross Products Division, Abbott Laboratories).

Infant formulas are liquids or reconstituted powders fed to infants and young children. They serve as substitutes for human milk. Infant formulas have a special role to play in the diets of infants because they are often the only source of nutrients for infants. Although breast-feeding is still the best nourishment for infants, infant formula is a close enough second that babies not only survive but thrive. Infant formula is becoming more and more increasingly close to breast milk.

A dairy product is described above. These products include, but are not limited to, whole milk, skim milk, fermented milk products such as yogurt or sour milk, cream, butter, condensed milk, dehydrated milk, coffee whitener, ice cream, cheese, whey products, lactose, etc.

In still another aspect this invention concerns a method of producing a flavonol-containing product which comprises: (a) cracking the seeds obtained from transformed plants of the invention to remove the meats from the hulls; and (b) flaking the meats obtained in step (a) to obtain the desired flake thickness.

### EXAMPLES

The present invention is further defined in the following Examples, in which parts and percentages are by weight and degrees are Celsius, unless otherwise stated. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1

### Construction of Plasmids for Transformation of Glycine max

The ability to decrease the amount of flavonols produced in soybean seed was tested. For this purpose, a recombinant construct (plasmid AC24) was prepared that would be capable of suppressing flavonol synthase. The methods used to prepare plasmid AC24 are described below.

Plasmid AC24 contains a seed-specific expression promoter followed by nucleotides that promote formation of a stem loop structure flanking nucleotides encoding a portion of the soybean flavonol synthase, and followed by a transcription termination signal. It is well understood by those skilled in the art that other sequences commonly used in molecular manipulations may be used here. These sequences may include any seed-specific promoter, any structure that promotes stem-loop formation, any polynucleotide encoding any portion of the gene or genes of interest inserted in sense or anti-sense orientation with respect to the promoter and stem-loop structure, and any termination signal. It is also well known by those skilled in the art that nucleotides promoting stem-loop formation are not always required for gene suppression. Plasmid AC24 was prepared as follows:

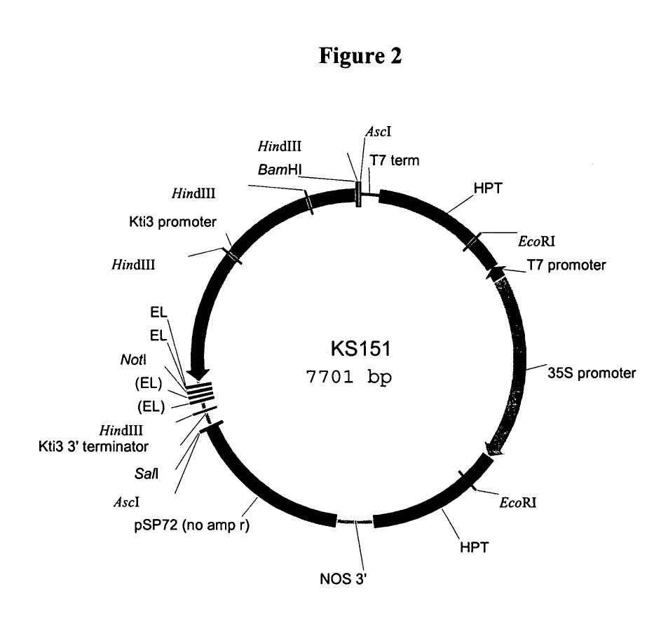

A polynucleotide encoding a portion of the soybean flavonol synthase was inserted into the seed-specific expression vector pKS151 to form plasmid AC24. Vector pKS151 is depicted in FIG. 2 and its nucleotide sequence is shown in SEQ ID NO:1. This vector has been described in PCT Publication WO 02/00904, published 3 Jan. 2002, and is derived from the commercially available vector pSP72 (Promega, Madison, Wis.).

To produce pKS151 vector pSP72 was modified by:
a) deleting the polynucleotide fragment corresponding to the beta lactamase coding region (nucleotides 1135 through 1995);
b) inserting a polynucleotide fragment encoding the hygromycin phosphotransferase enzyme (HPT) under the control of the bacterial T7 promoter and termination signals, for expression of the HPT enzyme in bacteria;
c) adding a polynucleotide comprising the cauliflower mosaic virus (CaMV) 35S promoter, a fragment encoding HPT, and the nopaline synthase (NOS) 3′end for constitutive expression of the HPT enzyme in plants; and
d) adding a polynucleotide comprising a unique Not 1 restriction endonuclease site surrounded by nucleotides that promote formation of a stem structure which are flanked by sequences corresponding to the kunitz trypsin inhibitor 3 (KTi3) promoter and KTi 3′ terminator.

Expression of HPT by two different promoters allows the selection for growth in the presence of hygromycin in bacterial and plant systems. The gene encoding KTi3 has been described (Jofuku and Goldberg (1989) Plant Cell 1:1079-1093). The KTi3 promoter in plasmid pKS151 includes about 2088 nucleotides upstream (5′) from the translation initiation codon, and the KTi3 terminator includes about 202 nucleotides downstream (3′) from the translation stop codon of KTi3. A unique Not I restriction endonuclease site can be found between the Kti3 5′ and 3′ regions. The Not I site is flanked by nucleotides that promote formation of a “stem-loop” structure when a polynucleotide from the gene of interest is inserted at the Not I site. This “stem-loop” structure will have the polynucleotide from the nucleic acid fragment of interest forming the loop. The stem structure is formed by two copies of 36 nucleotides at the 5′ end of the Not I site and an inverted repeat of the same two 36-nucleotide copies at the 3′ end.

A polynucleotide fragment encoding a portion of a soybean flavonol synthase was inserted in the Not I site of pKS151 to create plasmid AC24. The fragment corresponding to the flavonol synthase was obtained by PCR amplification using clone ssl.pk0057.d12 as template and primers flavonol synthase-Not1-sense (shown in SEQ ID NO:2) and flavonol synthase-Not1-antisense (shown in SEQ ID NO:3). Clone ssl.pk0057.d12 is identified in U.S. Pat. No. 6,380,464 as encoding an entire flavonol synthase. The nucleotide sequence of the cDNA insert in clone ssl.pk0057.d12 is shown in SEQ ID NO:4.

(SEQ ID NO:2)

5′- GCG GCC GCA TGG AGG TGC TAA GGG TG -3′

(SEQ ID NO:3)

5″- GCG GCC GCG CAT GTC ATC TTC ATT TG-3′

The amplification reaction was performed using advantage 2 polymerase and GC melt reagent (1 mM final concentration) and following the manufacturer's (Clontech, Palo Alto, Calif.) protocol. The resulting amplified DNA fragment was first cloned into TopoTA vector (Invitrogen, Carlsbad, Calif.). The fragment was liberated from the TopoTA vector by Not I digestion and was purified from an agarose gel using Qiagen Gel Purification Kit (Qiagen, Valencia, Calif.). The purified DNA fragment was inserted into the Not I site of vector pKS151 to produce the plasmid AC24.

### Example 2

### Transformation of Somatic Soybean Embryo Cultures and Regeneration of Soybean Plants

The ability to decrease the flavonol levels in transgenic soybean plants was tested by transforming soybean somatic embryo cultures with plasmid AC24, selecting transformants that grew in the presence of hygromycin, allowing plants to regenerate, and measuring the levels of kaempferol produced in seeds.

Soybean embryogenic suspension cultures were transformed with plasmid AC24 using particle gun bombardment.

The following stock solutions and media were used for transformation and regeneration of soybean plants:

Stock Solutions (per Liter):

MS Sulfate 100× stock: 37.0 g MgSO₄.7H₂O, 1.69 g MnSO₄.H₂O, 0.86 g ZnSO₄.7H₂O, 0.0025 g CuSO₄.5H₂O.

MS Halides 100× stock: 44.0 g CaCl₂.2H₂O, 0.083 g KI, 0.00125 g CoCl₂.6H₂O, 17.0 g KH₂PO₄, 0.62 g H₃BO₃, 0.025 g Na₂MoO₄.2H₂O, 3.724 g Na₂EDTA, 2.784 g FeSO₄.7H₂O.

B5 Vitamin stock: 100.0 g myo-inositol, 1.0 g nicotinic acid, 1.0 g pyridoxine HCl, 10.0 g thiamine.

2.4-D stock: 10 mg/mL

Media (per Liter):

SB55: 10 mL of each MS stock, 1 mL of B5 Vitamin stock, 0.8 g NH₄NO₃, 3.033 g KNO₃, 1 mL 2,4-D stock, 0.667 g asparagine, pH 5.7.

SB103: 1 pakage Murashige & Skoog salt mixture (Gibco, Carlsbad, Calif.), 60 g maltose, 2 g gelrite, pH 5.7.

SB71-1: B5 salts, 1 mL B5 vitamin stock, 30 g sucrose, 750 mg MgCl₂, 2 g gelrite, pH 5.7.

Soybean (of the Jack cultivar) embryogenic suspension cultures were maintained in 35 mL SB55 liquid media on a rotary shaker (150 rpm) at 28° C. with a mix of fluorescent and incandescent lights providing a 16 hour day, 8 hour night cycle. Cultures were subcultured every 2 to 3 weeks by inoculating approximately 35 mg of tissue into 35 mL of fresh liquid media.

Soybean embryonic suspension cultures were transformed by the method of particle gun bombardment (see Klein et al. (1987) Nature 327:70-73) using a DuPont Biolistic PDS1000/He instrument. Embryos were bombarded with plasmid pAC24 in a 1:10 molar ratio. Transformed lines were selected on medium containing hygromycin, and the presence of plasmid pAC24 was determined by PCR. Transgenic plants were generated from lines positive for the desired recombinant DNA fragments.

For bombardment, 5 μL of 1 μg/μL plasmid pAC24 DNA, 50 μL 2.5 M CaCl₂, and 20 μL 0.1 M spermidine were added to 50 μL of a 60 mg/mL 0.6 μm gold particle suspension. The particle preparation was agitated for 3 minutes, spun in a microfuge for 10 seconds and the supernatant removed. The DNA-coated gold particles were then washed once with 400 μL of 100% ethanol, resuspended in 40 μL of anhydrous ethanol, and sonicated three times for 1 second each. Five μL of the DNA-coated gold particles was then loaded on each macro carrier disk. Approximately 300 to 400 mg of two-week-old suspension culture was placed in an empty 60 mm×15 mm petri dish and the residual liquid removed from the tissue using a pipette. The tissue was placed about 3.5 inches away from the retaining screen and bombarded twice. Membrane rupture pressure was set at 1100 psi and the chamber was evacuated to −28 inches of Hg. Two plates were bombarded for each experiment and, following bombardment, the tissue was divided in half, placed back into liquid media, and cultured as described above.

Eleven days after bombardment, the liquid media was exchanged with fresh SB55 media containing 50 mg/mL hygromycin. The selective media was refreshed weekly. Seven weeks post bombardment, green, transformed tissue was observed growing from untransformed, necrotic embryogenic clusters. Isolated green tissue was removed and inoculated into individual flasks to generate new, clonally propagated, transformed embryogenic suspension cultures. Thus, each new line was treated as an independent transformation event. Soybean suspension cultures can be maintained as suspensions of embryos clustered in an immature developmental stage through subculture or can be regenerated into whole plants by maturation and germination of individual somatic embryos.

Transformed embryogenic clusters were removed from liquid culture and placed on SB103 solid agar media containing no hormones or antibiotics. Embryos were cultured for eight weeks at 26° C. with mixed fluorescent and incandescent lights on a 16 hour day, 8 hour night schedule. During this period, individual embryos were removed from the clusters and analyzed at various stages of embryo development. Lines that were resistant to hygromycin were assayed by PCR for the presence plasmid AC24. The presence of the plasmid AC24 was assayed by PCR amplification of DNA extracted from plant tissue. Primer Fsh-A (shown in SEQ ID NO:5) and Primer pS17 (shown in SEQ ID NO:6) were used in these amplifications.

(SEQ ID NO:5)

5′ - CTC ATT CTC TTC AAG CCC CAA CCC- 3′

(SEQ ID NO:6)

5′ - CCG ATT CTC CCA ACA TTG CTT ATT C - 3′

Somatic embryos became suitable for germination after eight weeks and were then removed from the maturation medium and dried in empty petri dishes for 1 to 5 days. The dried embryos were then planted in SB71-1 medium where they were allowed to germinate under the same lighting and germination conditions described above. Germinated embryos were transferred to sterile soil and grown to maturity. Seeds were harvested.

### Example 3

### Analysis of Flavonol Levels in Seeds of Transformants Containing Plasmid AC24

The quantity of kaempferol in seeds from transgenic plants comprising plasmid AC24 was assayed as follows. Five to eight seeds per transformant were combined and whole soybeans ground using an Adsit grinder (Adsit Co., Inc., Ft. Meade, Fla.). About 100 mg ground soybean was placed into a beater vial, accurately weighed, and a ¼ inch steel bead was added along with 1 mL of 60% acetonitrile. The mixture was agitated on a Genogrinder Model 2000 (SPEX Certiprep, Metuchen, N.J.) for 1 minute with the machine set at 1500 strokes per minute and then placed on an end-over-end tumbler for 1 hour. The vial was then placed in the Genogrinder for 1 minute with the machine set at 1500 strokes per minute and then centrifuged at 12,000 rpm for 5 minutes. The supernatant was then transferred to a 13×100 mm Pyrex tube fitted with a Teflon cap. One hundred μL of 10 mg/mL aqueous ascorbic acid was added to the extract and the solutions were mixed. Then, 120 μL of 12 N hydrochloric acid was added and the solutions were mixed. Tubes were placed in a heating block at 80° C. for 1 hour. After allowing the tube to cool to room temperature, the volume was measured and the tube was centrifuged at 3500 rpm for 10 minutes. Finally the supernatant was placed in an HPLC vial.

Kaempferol standards were prepared at the following concentrations 0.1, 0.25, 0.5, 1.0 and 2.0 PPM in 60% acetonitrile with 1 mg/mL ascorbic acid. Both samples and standards were analyzed by liquid chromatography/mass spectrometry (LC/MS) according to the following protocol. LC/MS was performed using a Waters (Milford, Mass.) 2690 Alliance HPLC interfaced with a ThermoQuest Finnigan (San Jose, Calif.) LCQ mass spectrometer. Samples were maintained at 20° C. prior to injection. A 10 μL sample was injected onto a Phenomenex (Torrance, Calif.) Luna C18 column (3μ, 4.6 mm×75 mm) maintained at 40° C. Compounds were eluted from the column at a flow rate of 0.8 mL/minute with 90% solvent A (0.1% formic acid in water) and 10% solvent B (0.1% formic acid in acetonitrile), followed by a linear gradient from 10% B to 20% from 0 to 0.5 minutes then held at 20% B from 0.5 to 6 minutes, followed by a linear gradient from 20% B to 50% from 6 to 8 minutes, then 50% B to 95% B from 10 to 12 minutes and then 90% A and 10% B from 12 to 17 minutes. The solvent flow was split post-column with 0.3 mL/minutes diverted to the mass spectrometer. The mass spectrometer was equipped with an ESI source set to scan m/z of 200 to 600 in positive ion mode. The capillary temperature 160° C., the sheath gas flow 60-psi, and the auxiliary gas flow 10 psi.

Analysis of R1 Seed from Transformation Events with Plasmid AC24

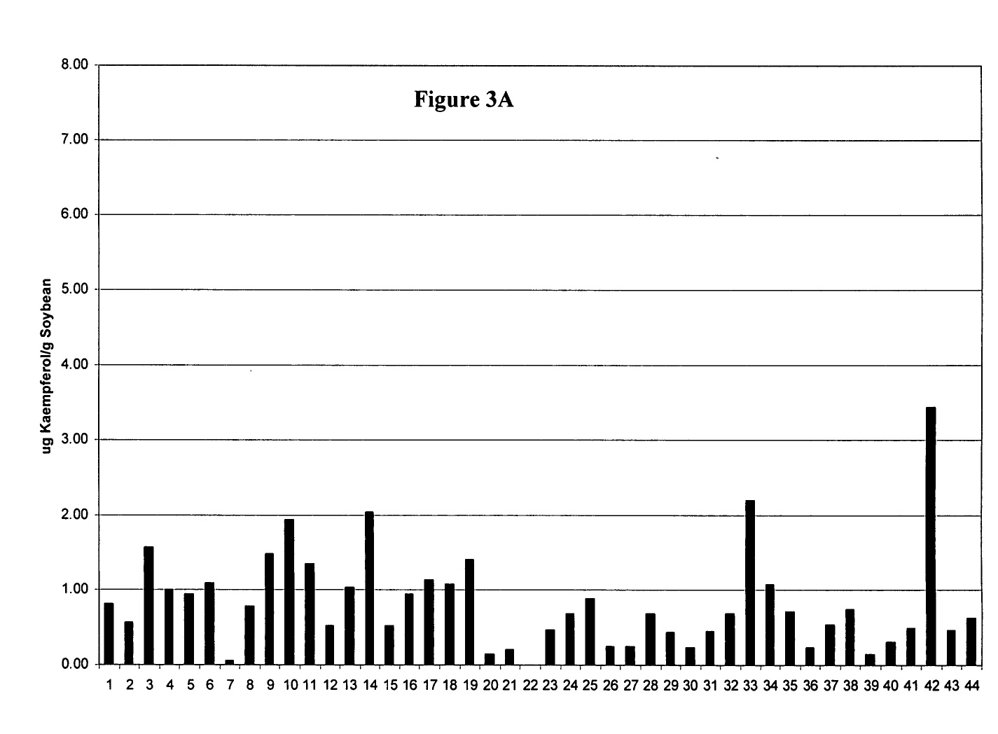

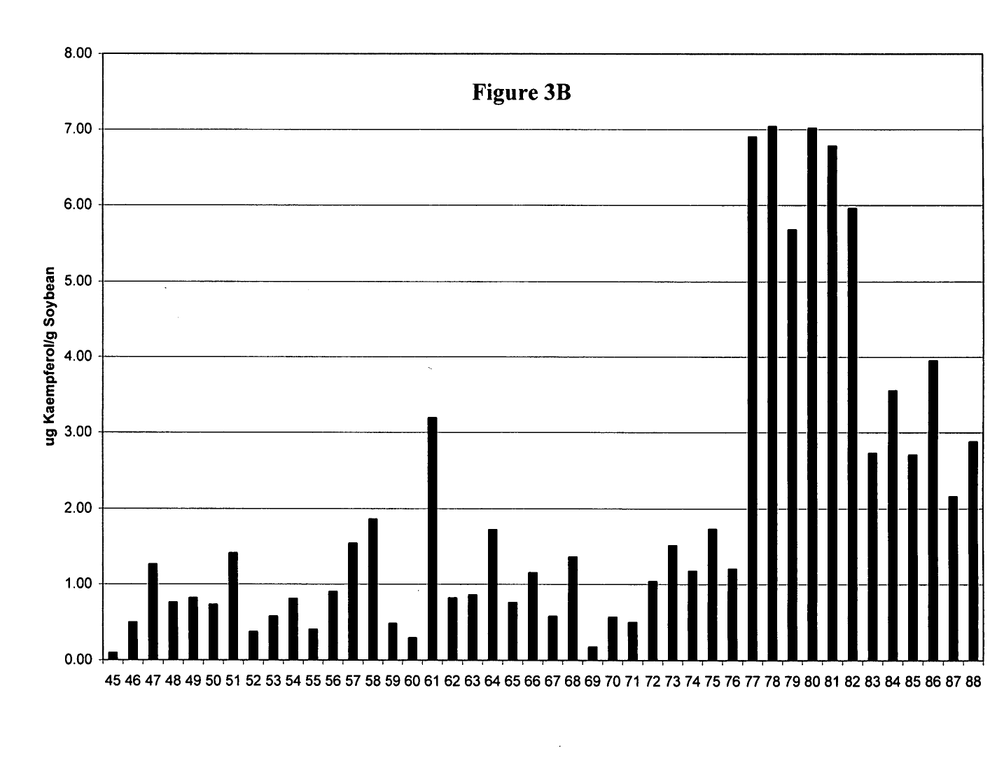

The levels of flavonols in 76 plants representing 45 independent events containing plasmid AC24 and in 12 non-transgenic control plants (6 from the Jack cultivar and 6 from the 93B41 cultivar) were assayed. Table 2 presents the level of kaempferol (in μg/g) in samples of transgenic seeds positive for plasmid AC24 or in controls. For ease of understanding, the plants from which the seeds are derived are numbered 1 through 88 in the figures, and the plant number indicated in the table. FIG. 3A presents the results obtained for plants 1-44 and FIG. 3B presents the results obtained for plants 45-88.

TABLE 2
Kaempferol Levels in R1 Seed Transgenic for Plasmid AC24

| # | Plant No. | μg Kaempferol/g Soybean |
|---|---|---|
| 1 | 3069-1-3-1 | 0.82 |
| 2 | 3069-1-3-2 | 0.57 |
| 3 | 3069-2-2-2 | 1.58 |
| 4 | 3069-2-2-3 | 1.01 |
| 5 | 3069-3-3-1 | 0.95 |
| 6 | 3069-3-3-3 | 1.10 |
| 7 | 3069-3-4-1 | 0.06 |
| 8 | 3069-3-5-2 | 0.79 |
| 9 | 3069-3-5-3 | 1.48 |
| 10 | 3069-4-4-1 | 1.94 |
| 11 | 3069-4-4-2 | 1.35 |
| 12 | 3069-5-1-2 | 0.53 |
| 13 | 3069-5-1-3 | 1.04 |
| 14 | 3069-5-3-1 | 2.04 |
| 15 | 3069-5-3-3 | 0.52 |
| 16 | 3069-5-4-2 | 0.95 |
| 17 | 3069-6-4-1 | 1.14 |
| 18 | 3069-6-4-2 | 1.08 |
| 19 | 3069-6-5-1 | 1.41 |
| 20 | 3075-1-6-1 | 0.14 |
| 21 | 3075-1-6-3 | 0.21 |
| 22 | 3075-5-7-2 | 0.00 |
| 23 | 3069-1-1-1 | 0.47 |
| 24 | 3069-1-1-2 | 0.68 |
| 25 | 3069-1-3-3 | 0.89 |
| 26 | 3069-1-4-1 | 0.25 |
| 27 | 3069-1-4-2 | 0.25 |
| 28 | 3069-3-1-1 | 0.69 |
| 29 | 3069-3-1-3 | 0.43 |
| 30 | 3069-3-2-1 | 0.23 |
| 31 | 3069-3-2-2 | 0.45 |
| 32 | 3069-3-4-2 | 0.68 |
| 33 | 3069-4-1-1 | 2.20 |
| 34 | 3069-4-1-2 | 1.08 |
| 35 | 3069-4-3-1 | 0.71 |
| 36 | 3069-4-3-2 | 0.23 |
| 37 | 3069-4-5-1 | 0.54 |
| 38 | 3069-4-5-2 | 0.74 |
| 39 | 3069-5-2-2 | 0.15 |
| 40 | 3069-6-1-1 | 0.31 |
| 41 | 3069-6-1-2 | 0.49 |
| 42 | 3069-6-2-3 | 3.44 |
| 43 | 3075-1-1-1 | 0.46 |
| 44 | 3075-1-1-2 | 0.62 |
| 45 | 3075-1-2-1 | 0.10 |
| 46 | 3075-1-2-2 | 0.50 |
| 47 | 3075-1-4-1 | 1.26 |
| 48 | 3075-1-4-2 | 0.76 |
| 49 | 3075-1-6-2 | 0.82 |
| 50 | 3075-2-2-1 | 0.73 |
| 51 | 3075-2-2-2 | 1.41 |
| 52 | 3075-2-7-1 | 0.37 |
| 53 | 3075-2-7-2 | 0.58 |
| 54 | 3075-2-8-1 | 0.81 |
| 55 | 3075-2-8-2 | 0.40 |
| 56 | 3075-2-9-1 | 0.90 |
| 57 | 3075-2-9-2 | 1.54 |
| 58 | 3075-3-1-1 | 1.86 |
| 59 | 3075-3-2-1 | 0.48 |
| 60 | 3075-3-3-1 | 0.29 |
| 61 | 3075-4-2-3 | 3.19 |
| 62 | 3075-4-5-1 | 0.82 |
| 63 | 3075-4-5-2 | 0.86 |
| 64 | 3075-4-6-1 | 1.72 |
| 65 | 3075-4-6-2 | 0.76 |
| 66 | 3075-5-1-1 | 1.15 |
| 67 | 3075-5-1-2 | 0.58 |
| 68 | 3075-5-2-1 | 1.36 |
| 69 | 3075-5-2-3 | 0.18 |
| 70 | 3075-5-4-1 | 0.57 |
| 71 | 3075-5-6-1 | 0.50 |
| 72 | 3075-5-6-2 | 1.04 |
| 73 | 3075-5-7-1 | 1.51 |
| 74 | 3075-5-7-3 | 1.17 |
| 75 | 3075-6-6-1 | 1.73 |
| 76 | 3075-6-6-3 | 1.20 |
| 77 | 93B41 | 6.90 |
| 78 | 93B41 | 7.04 |
| 79 | 93B41 | 5.68 |
| 80 | 93B41 | 7.02 |
| 81 | 93B41 | 6.78 |
| 82 | 93B41 | 5.96 |
| 83 | Jack | 2.73 |
| 84 | Jack | 3.55 |
| 85 | Jack | 2.71 |
| 86 | Jack | 3.95 |
| 87 | Jack | 2.16 |
| 88 | Jack | 2.88 |

As seen in the table above, suppression of flavonol synthase in transformed soybean plants produced lower levels of kaempferol than plants that were not transformed. Of 76 plants analyzed that were PCR positive for plasmid AC24, 49 plants representing 34 independent transformation events produced less than 1 μg kaempferol per gram soybean. Of these 49 plants, 10 plants representing 8 independent transformation events had less than 0.25 μg kaempferol per gram soybean. All control plants not containing the transgene produced more than 2 μg kaempferol per gram soybean.

Preparation of Soybean Milk Samples and Determination of Their Color

To determine if suppression of flavonol synthase results in soybeans that produce milk that is whiter, small samples of soybean milk were prepared as follows:

For each line, R1 seed samples weighing 0.9 g (+/−0.025 g) were transferred to a plastic scintillation vial. Hot distilled, deionized water was added to fill each vial to the top, the vial was capped tightly and boiled in a water bath. After boiling for 30 minutes the seeds were drained, transferred to fresh vials, and 15 mL of distilled, deionized water were added to each. Samples were homogenized completely using an Omni International (Warrenton, Va.) homogenizer and a 12 mm diameter (custom) generator probe with saw teeth and knife blade at a setting of 5 or 6 for approximately 1 minute. Each homogenized sample was transferred to a 50 mL round-bottom centrifuge tube and particulate removed by spinning in a DYNAC II centrifuge (Clay Adams division of Becton Dickinson & Co., Bedford, Mass.) for 1 minute at 1130 rpm. Using a pipette, approximately 6 mL of supernatant were transferred to a sample cup having a 5 mm ring. The Hunter L, a, b color (D65/10) and opacity (Y) were determined using a ColorFlex spectrocolorimeter (HunterLab, Reston, Va.) with 5 mm ring and white ceramic and black glass disks. “L” represents lightness (100-0), “a” redness (+) or greenness (−), and “b” yellowness (+) or blueness (−) of the sample on the Hunter L, a, b scale. Solids were measured using a CEM Corporation SMART System5 moisture solids analyzer. The measurements were made using the following settings: Power: 100%, Delta Wt: 0.5 mg, Delta time: 10 seconds, Max Time 4 minutes, Bias: 0%, Max Temp: 105° C., Result Range: Moist, Min Result 0%, Max Result 100%, Min Wt Range: 2.00 g, Max Wt Range: 4 g, Wt Comp: ON. The Whiteness index is calculated by using the formula: L-(3b).

Table 3 presents the results obtained for milk samples prepared from seeds produced by 5 transgenic soybean plants identified as containing plasmid AC24. Table also presents results for a Jack control. For each sample, Table 3 shows the transgenic plant ID number, the weight of soybean seed used in sample preparation (Wt), the L, a, and b values, the calculated white index, the opacity, and the percent solids. Table 3 also shows the average (Avg) and standard deviation (SD) of these numbers obtained for the 5 transgenic plants positive for plasmid AC24.

TABLE 3
Hunter L, a, b Color, and Opacity of Milk Samples
Prepared From Soybean Seeds of Transgenic Plants or Controls

| | | | | | | O- | |
|---|---|---|---|---|---|---|---|
| | | | | | White- | pac- | |
| | | | | | ness | ity | % |
| Plant ID | Wt | L | a | b | Index | (Y) | Solids |
| 3075-1-6-2 | 0.9 | 79.95 | −3.92 | 12.55 | 42.3 | 62.24 | 1.55 |
| 3075-1-6-3 | 0.89 | 79.49 | −4.24 | 13.56 | 38.81 | 60.28 | 1.58 |
| 3069-1-4-1 | 0.9 | 79.81 | −3.89 | 12.2 | 43.21 | 61.45 | 1.75 |
| 3069-3-2-1 | 0.896 | 79.32 | −4.08 | 13.68 | 38.28 | 61.63 | 1.77 |
| 3069-3-2-2 | 0.902 | 79.28 | −3.9 | 11.49 | 44.81 | 60.43 | 1.57 |
| | Avg | 79.57 | −4.01 | 12.7 | 41.48 | 61.21 | 1.64 |
| | SD | 0.298 | 0.15 | 0.927 | 2.83 | 0.83 | 0.11 |
| Control | 0.901 | 78.59 | −4.25 | 14.81 | 34.16 | 58.57 | 1.33 |

As seen in Table 3, the whiteness index of milk obtained from seeds derived from transgenic plants positive for plasmid AC24 is higher than that obtained for milk prepared from control seeds of the Jack cultivar.

1. A recombinant construct for reducing the level of flavonol in a flavonol-producing plant which comprises a promoter operably linked to a stem-loop structure wherein a nucleic acid sequence of at least 200 nucleotides and having at least 75% identity to SEQ ID NO:4 forms the loop part of the structure.

2. The recombinant construct of claim 1 wherein the promoter is a seed specific promoter.

3. The recombinant construct of claim 2 wherein said promoter is selected from the group consisting of a β-conglycinin promoter, a napin promoter, and a phaseolin promoter.

4. The recombinant construct of claim 1 or 2 wherein the stem consists essentially of SEQ ID NO:6.

5. The recombinant construct of claim 1 or 2 wherein the flavonol-producing plant is soybean.

6. The recombinant construct of claim 4 wherein the flavonol-producing plant is soybean.

7. A flavonol-producing plant comprising in its genome the recombinant construct of claim 1 or 2 wherein said plant has a reduced level of flavonol as compared to a plant which does not comprise the recombinant DNA construct of claim 1 or 2.

8. A flavonol-producing plant comprising in its genome the recombinant construct of claim 4.

9. The flavonol-producing plant of claim 7 wherein the flavonol-producing plant is soybean.

10. The flavonol-producing plant of claim 8 wherein the flavonol-producing plant is soybean.

11. Seeds or plant parts of the plant of claim 7.

12. Seeds or plant parts of the plant of claim 8.

13. A protein product having improved color wherein the product is obtained from the seeds or plant parts of claim 11.

14. A protein product having improved color wherein the product is obtained from the seeds or plant parts of claim 12.

15. The protein product of claim 13 wherein the product is selected from the group consisting of protein isolate, protein concentrate, meal, grits, full fat and defatted flours, textured proteins, textured flours, textured concentrates, and textured isolates.

16. The protein product of claim 14 wherein the product is selected from the group consisting of protein isolate, protein concentrate, meal, grits, full fat and defatted flours, textured proteins, textured flours, textured concentrates, and textured isolates.

17. A food product which has incorporated therein the protein product of claim 13.

18. A food product which has incorporated therein the protein product of claim 14.

19. A food product which has incorporated therein the protein product of claim 15.

20. A food product which has incorporated therein the protein product of claim 16.

21. A dairy product which has incorporated therein the protein product of claim 13.

22. A dairy product which has incorporated therein the protein product of claim 14.

23. A dairy product which has incorporated therein the protein product of claim 15.

24. A dairy product which has incorporated therein the protein product of claim 16.

25. A food bar which has incorporated therein the protein product of claim 13.

26. A food bar which has incorporated therein the protein product of claim 14.

27. A food bar which has incorporated therein the protein product of claim 15.

28. A food bar which has incorporated therein the protein product of claim 16.

29. A nutritional supplement which has incorporated therein the protein product of claim 13.

30. A nutritional supplement which has incorporated therein the protein product of claim 14.

31. A nutritional supplement which has incorporated therein the protein product of claim 15.

32. A nutritional supplement which has incorporated therein the protein product of claim 16.

33. A beverage which has incorporated therein the protein product of claim 13.

34. A beverage which has incorporated therein the protein product of claim 14.

35. A beverage which has incorporated therein the protein product of claim 15.

36. A beverage which has incorporated therein the protein product of claim 16.

37. A method for reducing the level of flavonol in a flavonol producing plant which comprises
a) transforming an flavonol producing plant cell with a recombinant construct comprising a promoter operably linked to a stem-loop structure wherein a nucleic acid sequence of at least 200 nucleotides and having at least 75% identity to SEQ ID NO:4 forms the loop part of the structure;
b) regenerating a transformed plant from the plant cell of step (a); and
c) evaluating the transformed plant obtained from step (b) for a reduced level of flavonol.

38. The method of claim 37 wherein the promoter is a seed specific promoter.

39. The method of claim 38 wherein said promoter is selected from the group consisting of a β-conglycinin promoter, a napin promoter, and a phaseolin promoter

40. The method of claim 37 or 38 wherein the stem consists essentially of SEQ ID NO:6.

41. The method of claim 37 or 38 wherein the flavonol-producing plant is soybean.

42. The method of claim 40 wherein the flavonol-producing plant is soybean.

43. A flavonol-producing plant made by the method of claim 37 or 38.

44. A flavonol-producing plant made by the method of claim 40.

45. Seeds or plant parts obtained from the flavonol-producing plant of claim 43.

46. Seeds or plant parts obtained from the flavonol-producing plant of claim 44.

47. A protein product having improved color obtained from the seeds or plant parts of claim 45.

48. A protein product having improved color obtained from the seeds or plant parts of claim 46.

49. The protein product of claim 47 wherein the product is selected from the group consisting of protein isolate, protein concentrate, meal, grits, full fat and defatted flours, textured proteins, textured flours, textured concentrates, and textured isolates.

50. The protein product of claim 48 wherein the product is selected from the group consisting of protein isolate, protein concentrate, meal, grits, full fat and defatted flours, textured proteins, textured flours, textured concentrates, and textured isolates.

51. A food product which has incorporated therein the protein product of claim 47.

52. A food product which has incorporated therein the protein product of claim 48.

53. A food product which has incorporated therein the protein product of claim 49.

54. A food product which has incorporated therein the protein product of claim 50.

55. A dairy product which has incorporated therein the protein product of claim 47.

56. A dairy product which has incorporated therein the protein product of claim 48.

57. A dairy product which has incorporated therein the protein product of claim 49.

58. A dairy product which has incorporated therein the protein product of claim 50.

59. A food bar which has incorporated therein the protein product of claim 47.

60. A food bar which has incorporated therein the protein product of claim 48.

61. A food bar which has incorporated therein the protein product of claim 49.

62. A food bar which has incorporated therein the protein product of claim 50.

63. A nutritional supplement which has incorporated therein the protein product of claim 47.

64. A nutritional supplement which has incorporated therein the protein product of claim 48.

65. A nutritional supplement which has incorporated therein the protein product of claim 49.

66. A nutritional supplement which has incorporated therein the protein product of claim 50.

67. A beverage which has incorporated therein the protein product of claim 47.

68. A beverage which has incorporated therein the protein product of claim 48.

69. A beverage which has incorporated therein the protein product of claim 49.

70. A beverage which has incorporated therein the protein product of claim 50.


SEQUENCE LISTING (7 sequences)

SEQ ID NO: 1 - DNA, 7680 bp, Plasmid pKS151
  misc_feature (6516)..(6516): n = a, c, g, or t
cgcgcccgatcatccggatatagttcctcctttcagcaaaaaacccctcaagacccgtttagaggccccaaggggttatgctagttattgctcagcggtggcagcagccaactcagcttcctttcgggctttgttagcagccggatcgatccaagctgtacctcactattcctttgccctcggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatagcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggcttttccatgggtatatctccttcttaaagttaaacaaaattatttctagagggaaaccgttgtggtctccctatagtgagtcgtattaatttcgcgggatcgagatcgatccaattccaatcccacaaaaatctgagcttaacagcacagttgctcctctcagagcagaatcgggtattcaacaccctcatatcaactactacgttgtgtataacggtccacatgccggtatatacgatgactggggttgtacaaaggcggcaacaaacggcgttcccggagttgcacacaagaaatttgccactattacagaggcaagagcagcagctgacgcgtacacaacaagtcagcaaacagacaggttgaacttcatccccaaaggagaagctcaactcaagcccaagagctttgctaaggccctaacaagcccaccaaagcaaaaagcccactggctcacgctaggaaccaaaaggcccagcagtgatccagccccaaaagagatctcctttgccccggagattacaatggacgatttcctctatctttacgatctaggaaggaagttcgaaggtgaaggtgacgacactatgttcaccactgataatgagaaggttagcctcttcaatttcagaaagaatgctgacccacagatggttagagaggcctacgcagcaggtctcatcaagacgatctacccgagtaacaatctccaggagatcaaataccttcccaagaaggttaaagatgcagtcaaaagattcaggactaattgcatcaagaacacagagaaagacatatttctcaagatcagaagtactattccagtatggacgattcaaggcttgcttcataaaccaaggcaagtaatagagattggagtctctaaaaaggtagttcctactgaatctaaggccatgcatggagtctaagattcaaatcgaggatctaacagaactcgccgtgaagactggcgaacagttcatacagagtcttttacgactcaatgacaagaagaaaatcttcgtcaacatggtggagcacgacactctggtctactccaaaaatgtcaaagatacagtctcagaagaccaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatgcctctgccgacagtggtcccaaagatggacccccacccacgaggagcatcgtggaaaaagaagacgttccaaccacgtcttcaaagcaagtggattgatgtgacatctccactgacgtaagggatgacgcacaatcccactatccttcgcaagacccttcctctatataaggaagttcatttcatttggagaggacacgctcgagctcatttctctattacttcagccataacaaaagaactcttttctcttcttattaaaccatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacctaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatgtcgaatctgatcaacctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatggacatattgtcgttagaacgcggctacaattaatacataaccttatgtatcatacacatacgatttaggtgacactatagaacggcgcgccgtcgacggatataatgagccgtaaacaaagatgattaagtagtaattaatacgtactagtaaaagtggcaaaagataacgagaaagaaccaatttctttgcattcggccttagcggaaggcatatataagctttgattattttatttagtgtaatgatttcgtacaaccaaagcatttatttagtactctcacacttgtgtcgcggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccgcttggggggctatggaagactttcttagttagttgtgtgaataagcaatgttgggagaatcgggactacttataggataggaataaaacagaaaagtattaagtgctaatgaaatatttagactgataattaaaatcttcacgtatgtccacttgatataaaaacgtcaggaataaaggaagtacagtagaatttaaaggtactctttttatatatacccgtgttctctttttggctagctagttgcataaaaaataatctatatttttatcattattttaaatatcttatgagatggtaaatatttatcataattttttttactattatttattatttgtgtgtgtaatacatatagaagttaattacaaattttatttactttttcattattttgatatgattcaccattaatttagtgttattatttataatagttcattttaatctttttgtatatattatgcgtgcagtacttttttcctacatataactactattacattttatttatataatatttttattaatgaattttcgtgataatatgtaatattgttcattattatttcagattttttaaaaatatttgtgttattatttatgaaatatgtaatttttttagtatttgattttatgatgataaagtgttctaaattcaaaagaagggggaaagcgtaaacattaaaaaacgtcatcaaacaaaaacaaaatcttgttaataaagataaaactgtttgttttgatcactgttatttcgtaatataaaaacattatttatatttatattgttgacaaccaaatttgcctatcaaatctaaccaatataatgcatgcgtggcaggtaatgtactaccatgaacttaagtcatgacataataaaccgtgaatctgaccaatgcatgtacctanctaaattgtatttgtgacacgaagcaaatgattcaattcacaatggagatgggaaacaaataatgaagaacccagaactaagaaagcttttctgaaaaataaaataaaggcaatgtcaaaagtatactgcatcatcagtccagaaagcacatgatatttttttatcagtatcaatgcagctagttttattttacaatatcgatatagctagtttaaatatattgcagctagatttataaatatttgtgttattatttatcatttgtgtaatcctgtttttagtattttagtttatatatgatgataatgtattccaaatttaaaagaagggaaataaatttaaacaagaaaaaaagtcatcaaacaaaaaacaaatgaaagggtggaaagatgttaccatgtaatgtgaatgttacagtatttcttttattatagagttaacaaattaactaatatgattttgttaataatgataaaatattttttttattattatttcataatataaaaatagtttacttaatataaaaaaaattctatcgttcacaacaaagttggccacctaatttaaccatgcatgtacccatggaccatattaggtaaccatcaaacctgatgaagagataaagagatgaagacttaagtcataacacaaaaccataaaaaacaaaaatacaatcaaccgtcaatctgaccaatgcatgaaaaagctgcaatagtgagtggcgacacaaagcacatgattttcttacaacggagataaaaccaaaaaaatatttcatgaacaacctagaacaaataaagcttttatataataaatatataaataaataaaggctatggaataatatacttcaatatatttggattaaataaattgttggcggggttgatatatttatacacacctaaagtcacttcaatctcattttcacttaacttttattttttttttctttttatttatcataaagagaatattgataatatactttttaacatatttttatgacattttttattggtgaaaacttattaaaaatcataaattttgtaagttagatttatttaaagagttcctcttcttattttaaattttttaataaatttttaaataactaaaatttgtgttaaaaatgttaaaaaatgtgttattaacccttc

SEQ ID NO: 2 - DNA, 26 bp, Artificial
  misc_feature (1)..(26): Oligonucleotide primer flavonol synthase-Not1-sense
gcggccgcatggaggtgctaagggtg

SEQ ID NO: 3 - DNA, 26 bp, Artificial
  misc_feature (1)..(26): Oligonucleotide primer flavonol synthase-Not1-antisense
gcggccgcgcatgtcatcttcatttg

SEQ ID NO: 4 - DNA, 1293 bp, Glycine max
aatcaacacacacaaacacaacaacatatggaggtgctaagggtgcaaaccatagcttccaaatccaaagatgctgccatcccagccatgtttgttagggcagagacagagcaaccaggcatcacaaccgttcaaggggtgaaccttgaggtgccaattattgattttagtgacccagatgaagggaaagtggtgcatgagattttggaggcaagtagggactggggcatgttccaaattgtgaaccatgacatacctagtgatgttataagaaagttgcaaagtgttgggaaaatgttctttgagttgccacaagaggaaaaagagttgattgctaagcctgctgggtctgattctattgaagggtatggcacaaagcttcagaaagaggtgaatggcaagaaagggtgggtggatcatttgttccacattgtgtggcctccttcctccatcaactacagtttctggccccaaaaccccccttcttacagggaagttaatgaggaatattgcaagcacctaagaggagtggtagacaaattgttcaaaagtatgtcggtagggttggggcttgaagagaatgagctaaaggagggtgcaaatgaagatgacatgcattatcttttaaaaatcaattattacccaccttgtccatgtcctgatctggtcttgggtgtgccaccacacacagacatgtcctacctcacaattctggtgcctaacgaggtgcagggccttcaagcatgtagggatggccattggtacgatgttaagtatgtccccaatgccctcgttattcacattggcgaccaaatggagatactgagcaatggaaaatataaggcagtttttcacagaacaacagtgaacaaagatgagacaagaatgtcgtggcccgtgttcatagaacccaaaaaggaacaagaagttggtcctcacccaaagttggttaaccaagacaatccaccaaaatacaaaaccaagaaatacaaggattatgcttattgtaagctcaataagatccctcaatgaatgaagtgggcactacgagataattatagctcatggtttctctgttttgttatttaatttatggaatgaaagtgtgacttgtgggaagtagattaaataagatctgtgactaatgttggtttctctgttttgttatttaatttatggaatgaaagtgtgacttgtgggaagtagattaaataagatctgtgactaatgttggattaatcttatgcttttgaagtaaataaagatgacaagaaacagtttgtctgttctgtt

SEQ ID NO: 5 - DNA, 24 bp, Artificial
  misc_feature (1)..(24): Oligonucleotide primer Fsh-A
ctcattctcttcaagccccaaccc

SEQ ID NO: 6 - DNA, 25 bp, Artificial
  misc_feature (1)..(25): Oligonucleotide primer pS17
ccgattctcccaacattgcttattc

SEQ ID NO: 7 - DNA, 154 bp, Artificial
  misc_feature (1)..(154): Artificial sequence containing a NotI site flanked by two 36-nucleotide repeats and having an EagI site at each end.
cggccggagctggtcatctcgctcatcgtcgagtcggcggccggagctggtcatctcgctcatcgtcgagtcggcggccgccgactcgacgatgagcgagatgaccagctccggccgccgactcgacgatgagcgagatgaccagctccggccg